(19) United States
(12) Patent Application Publication
(10) Pub. No.: US 2005/0228297 A1
(43) Pub. Date: Oct. 13, 2005

(54) Wrist-worn System for Measuring Blood Pressure

(76) Inventors: Matthew J. Banet, Del Mar, CA (US); Henk Visser, San Diego, CA (US); Robert Murad, San Diego, CA (US)

Correspondence Address: MORRISON ULMAN, WOODSIDE IP GROUP 1900 EMBARCADERO ROAD SUITE 209, PALO ALTO, CA, 94303-3327, US

(21) Appl. No.: 10/709,015

(22) Filed: Apr. 7, 2004

(51) Int. Cl. A61B 5/02

(52) U.S. Cl. 600/485; 128/903

(57) ABSTRACT

The invention provides a device that measures a patient's blood pressure without using an inflatable cuff. The device includes an optical module featuring an optical source and an optical detector; a flexible, thin-film pressure sensor; and a processing module, configured to receive and process information to calculate time-dependent blood pressure data and send the data to a web site using wireless data transmission techniques.

### BACKGROUND OF INVENTION

1. Field of the Invention

The present invention features a cuffless blood-pressure monitor that wirelessly transmits data to an Internet-based system.

2. Description of Related Art

Blood within a patient's body is characterized by a base-line pressure value, called the diastolic pressure. Diastolic pressure indicates a pressure in an artery when the blood it contains is static. A heartbeat forces a time-dependent volume of blood through the artery, causing the baseline pressure to increase in a pulse-like manner to a value called the systolic pressure. The systolic pressure indicates a maximum pressure in a portion of the artery that contains a flowing volume of blood.

Pressure in the artery periodically increases from the diastolic pressure to the systolic pressure in a pulsatile manner, with each pulse corresponding to a single heartbeat. Blood pressure then returns to the diastolic pressure when the flowing pulse of blood passes through the artery.

Both invasive and non-invasive devices can measure a patient's systolic and diastolic blood pressure. A noninvasive medical device called a sphygmomanometer measures a patient's blood pressure using an inflatable cuff and a sensor (e.g., a stethoscope) that detects blood flow by listening for sounds called the Korotkoff sounds. During a measurement, a medical professional typically places the cuff around the patient's arm and inflates it to a pressure that exceeds the systolic blood pressure. The medical professional then incrementally reduces pressure in the cuff while listening for flowing blood with the stethoscope. The pressure value at which blood first begins to flow past the deflating cuff, indicated by a Korotkoff sound, is the systolic pressure. The stethoscope monitors this pressure by detecting strong, periodic acoustic “beats” or “taps” indicating that the blood is flowing past the cuff (i.e., the systolic pressure barely exceeds the cuff pressure). The minimum pressure in the cuff that restricts blood flow, as detected by the stethoscope, is the diastolic pressure. The stethoscope monitors this pressure by detecting another Korotkoff sound, in this case a “leveling off” or disappearance in the acoustic magnitude of the periodic beats, indicating that the cuff no longer restricts blood flow (i.e., the diastolic pressure barely exceeds the cuff pressure).

Low-cost, automated devices measure blood pressure using an inflatable cuff and an automated acoustic or pressure sensor that measures blood flow. These devices typically feature cuffs fitted to measure blood pressure in a patient's wrist, arm or finger. During a measurement, the cuff automatically inflates and then incrementally deflates while the automated sensor monitors blood flow. A microcontroller in the automated device then calculates blood pressure. Cuff-based blood-pressure measurements such as these typically only determine the systolic and diastolic blood pressures; they do not measure dynamic, time-dependent blood pressure.

Time-dependent blood pressure can be measured with an invasive device, called a tonometer. The tonometer is typically inserted into an opening in a patient's skin and features a component that compresses an artery against a portion of bone. A pressure sensor within the device then measures blood pressure in the form of a time-dependent waveform. The waveform features a baseline that indicates the diastolic pressure, and time-dependent pulses, each corresponding to individual heartbeats. The maximum value of each pulse is the systolic pressure. The rising and falling edges of each pulse correspond to pressure values that lie between the systolic and diastolic pressures.

Data indicating blood pressure are most accurately measured during a patient's appointment with a medical professional, such as a doctor or a nurse. Once measured, the medical professional manually records these data in either a written or electronic file. Appointments typically take place a few times each year. Unfortunately, in some cases, patients experience “white coat syndrome” where anxiety during the appointment affects the blood pressure that is measured. For example, white coat syndrome can elevate a patient's heart rate and blood pressure; this, in turn, can lead to an inaccurate diagnosis.

Some medical devices for measuring blood pressure and other vital signs include systems for transmitting data from a remote site, such as the patient's home, to a central database. These systems can include a conventional computer modem that transmits data through a telephone line to the database. Or alternatively they can include a wireless transmitter, such as a cellular telephone, which wirelessly transmits the data through a wireless network.

### BRIEF DESCRIPTION OF DRAWINGS

The features and advantages of the present invention can be understood by reference to the following detailed description taken with the drawings, in which:

FIG. 1 is a schematic side view of the cuffless blood-pressure monitor of the invention, featuring a “watch” component and a wireless hub;

FIG. 2A is a top view of the watch component of FIG. 1, featuring finger and wrist-mounted modules;

FIG. 2B is a side view of the wireless hub of FIG. 1;

FIG. 3 is a schematic diagram of the electrical components of the watch component and wireless hub used in the blood-pressure monitor of FIGS. 1, 2A, and 2B;

FIG. 4 is a schematic view of an Internet-based system, coupled with the blood-pressure monitor of FIG. 1, that transmits blood-pressure data through a wireless network to an Internet-accessible host computer system;

FIG. 5 is a graph of optical and pressure waveforms, measured by a watch component of the invention, that are processed to determine blood pressure; and

FIG. 6 is a graph of time-dependent blood pressure measured from a patient by processing the time-dependent waveforms of FIG. 5.

### DETAILED DESCRIPTION

The following description refers to the accompanying drawings that illustrate certain embodiments of the present invention. Other embodiments are possible and modifications may be made to the embodiments without departing from the spirit and scope of the invention. Therefore, the following detailed description is not meant to limit the present invention. Rather, the scope of the present invention is defined by the appended claims.

An aspect of the invention is to provide a cuffless, wrist-worn blood-pressure monitor that features a form factor similar to a common watch. The monitor typically includes two parts: a watch component that measures blood pressure, and a separate wireless hub that sends this and other information to an Internet-accessible website for viewing and analysis. The watch component features individual sensors that measure optical and pressure waveforms, and a microcontroller that analyzes these waveforms to determine beat-to-beat blood pressure without using a constrictive cuff. A short-range wireless transmitter (using, e.g., a Bluetooth™ protocol) within the watch component sends this information to a matched receiver in the wireless hub. Additionally the hub includes a long-range wireless transmitter (e.g., a radio modem) that sends the blood-pressure information through a wireless network to an Internet-based website.

Patients can order the monitor using a separate page in the Internet-based website and it use continuously for a short (e.g. 1 month) period of time. During this time information is periodically sent (e.g., every 15 minutes) to the website, where software monitors the incoming data and transmits summary reports to the patient. When the monitoring period is complete the patient returns the monitor.

Specifically, in one aspect, the invention provides a blood-pressure monitoring device featuring: 1) a thin-film, pressure-monitoring module containing a pressure-sensitive region; 2) an optical module containing an optical source and an optical detector; and 3) a microprocessor configured to receive and process information from the thin-film, pressure-monitoring module and the optical module to determine blood pressure.

The pressure-sensitive region within the thin-film, pressure-monitoring module typically includes a material characterized by pressure-dependent electrical properties, e.g. a resistance that varies with applied pressure. This component can include a plastic film that encases the pressure-sensitive region. Within the optical module, the optical source is typically a laser or a light-emitting diode, and the optical detector is a photodiode. In typical embodiments, a finger-mounted component, such as an annular ring, houses the optical module. A wrist-mounted component, typically having a form factor similar to a conventional watch, houses the thin-film pressure-monitoring module.

The blood-pressure monitoring device typically includes a short-range wireless transmitter operating on a wireless protocol based on Bluetooth™, part-15, or 802.11. In this case, “part-15” refers to a conventional low-power, spread-spectrum, short-range wireless protocol, such as that used in cordless telephones. In typical embodiments, the short-range wireless transmitter sends information to an external, secondary wireless component that includes a short-range wireless receiver (also operating a Bluetooth™, part-15, or 802.11 wireless protocol) and a long-range wireless transmitter. The long-range wireless transmitter transmits information over a terrestrial, satellite, or 802.11-based wireless network. Suitable networks include those operating at least one of the following protocols: CDMA, GSM, GPRS, Mobitex, DataTac, iDEN, and analogs and derivatives thereof.

To measure blood pressure, the pressure-monitoring module generates a pressure waveform, and the optical module generates an optical waveform. The microprocessor runs computer-readable code that processes both the optical and pressure waveforms to determine blood pressure. The term “microprocessor” means a silicon-based microprocessor or microcontroller that can run compiled computer code to perform mathematical operations on data stored in a memory. Examples include ARM7 or ARM9 microprocessors manufactured by a number of different companies; AVR 8-bit RISC microcontrollers manufactured by Atmel; PIC CPUs manufactured by Microchip Technology Inc.; and high-end microprocessors manufactured by Intel and AMD.

In the above-described system, the term “wireless network” refers to a standard wireless communication network. These networks, described in more detail below, connect a wireless transmitter or a silicon-based chipset to the Internet-based software piece.

The invention has many advantages. In particular, it allows patients to conduct a low-cost, comprehensive, real-time monitoring of their blood pressure. Information can be viewed using an Internet-based website, using a personal computer, or simply by viewing a display on the monitor. Data measured several times each day provide a relatively comprehensive data set compared to that measured during medical appointments separated by several weeks or even months. This allows both the patient and medical professional to observe trends in the data, such as a gradual increase or decrease in blood pressure, which may indicate a medical condition. The invention also minimizes effects of white coat syndrome since the monitor automatically makes measurements with basically no discomfort; measurements are made at the patient's home or work rather than in a medical office.

Real-time, automatic blood pressure measurements, followed by wireless transmission of the data, are only practical with a non-invasive, cuffless monitor like that of the present invention. Measurements can be made completely unobtrusive to the patient. And the monitor alleviates conditions, such as an uncomfortable or poorly fitting cuff, that can erroneously affect a blood-pressure measurement.

The monitor can also measure pulse oximetry to characterize the patient's heart rate and blood oxygen saturation using the same optical system for the blood-pressure measurement. These data can be wirelessly transmitted and used to further diagnose the patient's cardiac condition.

The monitor is small, easily worn by the patient during periods of exercise or day-to-day activities, and makes a non-invasive blood-pressure measurement in a matter of seconds. Measurements can be made with no effect on the patient. An on-board or remote processor can analyze the time-dependent measurements to generate statistics on a patient's blood pressure (e.g., average pressures, standard deviation, beat-to-beat pressure variations) that are not available with conventional devices that only measure systolic and diastolic blood pressure at isolated times.

Ultimately the wireless, internet-based blood pressure-monitoring system described herein provides an in-depth, cost-effective mechanism to evaluate a patient's cardiac condition. Certain cardiac conditions can be controlled, and in some cases predicted, before they actually occur. Moreover, data from the patient can be collected and analyzed while the patient participates in their normal, day-to-day activities. This provides a relatively comprehensive diagnosis that is not possible using a conventional medical-diagnostic system.

The resulting data, of course, have many uses for patients, medical professional, insurance companies, pharmaceutical agencies conducting clinical trials, and organizations for home-health monitoring.

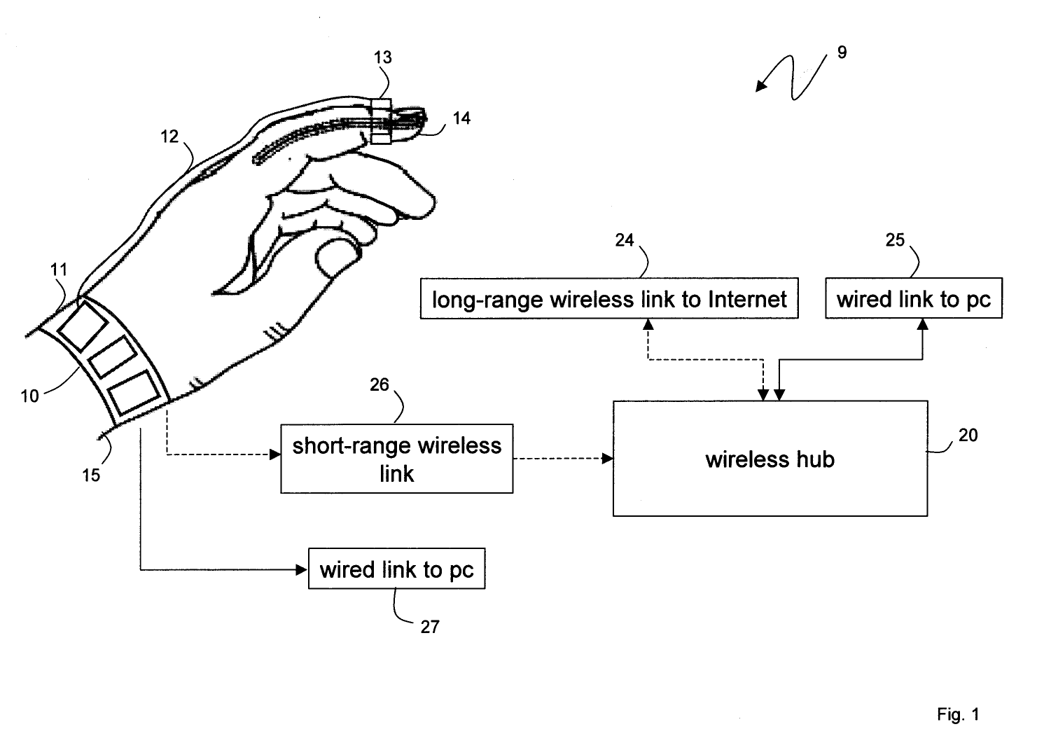

FIG. 1 shows an optical, cuffless blood-pressure monitor 9 according to the invention that measures a patient's real-time, beat-to-beat blood pressure. The monitor 9 features a watch component 10 that measures blood pressure without using a cuff, and a wireless hub 20 that receives and transmits this information to an Internet-accessible website. The watch component 10 features an optical finger-mounted module 13 that attaches to a patient's index finger 14, and a wrist-mounted module 11 that attaches to an area 15 of the patient's wrist where a watch is typically worn. A cable 12 provides an electrical connection between the finger-mounted 13 and wrist-mounted 11 modules. During operation, the finger-mounted module 13 measures an optical “waveform” and the wrist-mounted module measures a pressure “waveform” as described in detail below. Once these waveforms are measured, the watch component 10 processes them to determine diastolic and systolic blood pressure, real-time beat-to-beat blood pressure, heart rate, and pulse oximetry. The watch component 10 transfers this information using a short-range wireless link 26 to the wireless hub 20. The hub 20 receives the information and, in turn, sends it over a long-range wireless link 24 to an Internet-accessible website. In order to send information directly to a personal computer, both the watch component 10 and the wireless hub 20 include wired links 25, 27 (e.g., a serial cable connected to a serial port) to a personal computer.

Software programs associated with the Internet-accessible website and the personal computer analyze the blood pressure, and heart rate, and pulse oximetry values to characterize the patient's cardiac condition. These programs, for example, may provide a report that features statistical analysis of these data to determine averages, data displayed in a graphical format, trends, and comparisons to doctor-recommended values.

The blood-pressure monitor 9 measures cardiac information non-invasively with basically no inconvenience to the patient. This means information can be measured in real time and throughout the day, e.g., while the patient is working, sleeping, or exercising. For example, during work or sleep, the wireless hub 20 rests near the patient (e.g. on a desktop), while during exercise it attaches to the patient's belt. In this way, the blood-pressure monitor 9, combined with the above-described software programs, provides an extensive, thorough analysis of the patient's cardiac condition. Such analysis is advantageous compared to conventional blood-pressure measurements, which are typically made sporadically with an uncomfortable cuff, and thus may not accurately represent the patient's cardiac condition.

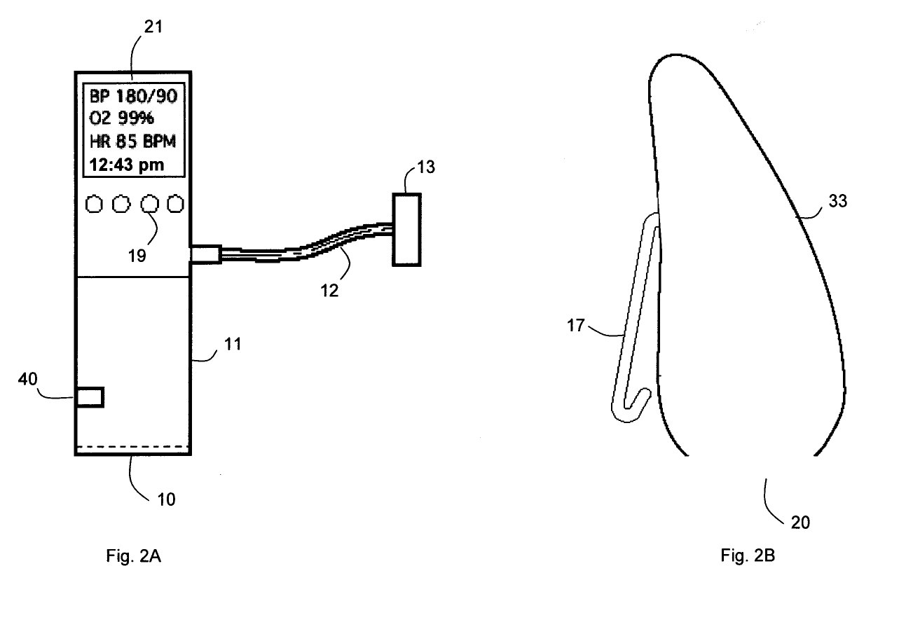

FIGS. 2A and 2B show, respectively, mechanical drawings of the watch component 10 and wireless hub 20. The watch component 10 features a wrist-mounted module 11 that looks similar to a conventional watch, and includes an LCD display 21 that shows, for example, diastolic and systolic blood pressure values, pulse oximetry, heart rate, and the time of day. Using a series of buttons 19, the patient can select additional functions, such as historical and statistical analysis, or a graphical display, of this information. The finger-mounted module 13 looks like a conventional finger ring, and connects to the wrist-mounted module 11 using a thin, transparent cable 12 that, during use, rests on a top portion of the patient's wrist. The wrist-mounted module 11 additionally includes a serial port 40 having a form similar to a stereo-jack connector that downloads information to a personal computer using an appropriate cable.

The wireless hub 20 features a discrete plastic case 33 that houses its electronics and is small enough to be placed in a purse or rest on a desktop. The case 33 includes a clip 17 that attaches, e.g., to the patient's belt so it can be worn daily or during exercise. Using the short-range wireless link, the wireless hub 20 receives information when it comes within about twenty feet of the watch component, and then automatically transmits the information through a wireless network as described in more detail below.

When a distance greater than twenty feet separates the hub 20, the watch component 10 simply stores information in memory and continues to make measurements. The watch component automatically transmits all the stored information (along with a time/date stamp) when it comes in proximity to the hub 20, which then transmits the information through the wireless network.

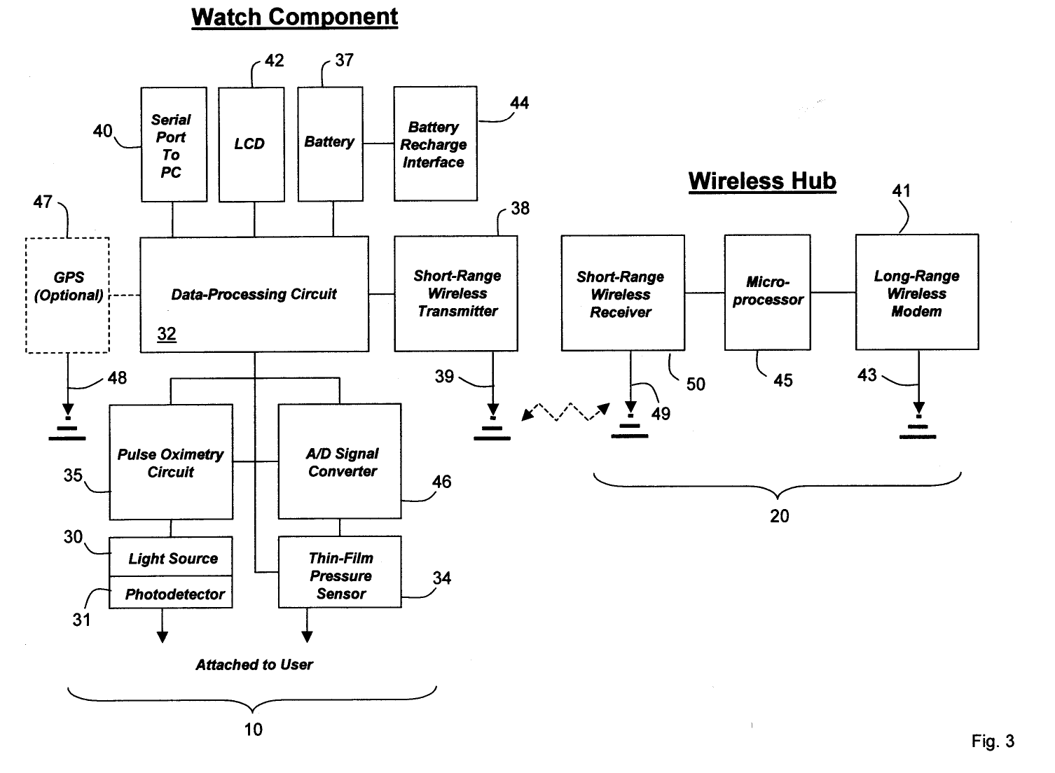

FIG. 3 shows in detail electronic components featured in both the watch component 10 and the wireless hub 20. To generate the optical waveform, the watch component 10 includes a light source 30 and a photodetector 31 within the finger-mounted module. The light source 30 typically includes light-emitting diodes that generate both red (λ˜630 nm) and infrared (λ˜900 nm) radiation. As the heart pumps blood through the patient's finger, blood cells absorb and transmit varying amounts of the red and infrared radiation depending on how much oxygen binds to the cells″ hemoglobin. The photodetector 31 detects transmission at the red and infrared wavelengths, and in response generates a radiation-induced current that travels through a cable to a pulse-oximetry circuit 35 embedded within the wrist-worn module. The pulse-oximetry circuit 35 connects to an analog-to-digital signal converter 46 that converts the radiation-induced current into the time-dependent optical waveform, which is then sent back to the pulse-oximetry circuit 35 and analyzed to determine both heart rate and the pulse oximetry value.

The wrist-mounted module additionally includes a thin-film pressure sensor 34 that includes a pressure-sensitive region. This region features a pressure-sensitive film characterized by an electrical resistance that varies with the amount of applied pressure. Such sensors, e.g., include the ELF sensor manufactured by Tekscan of South Boston, Mass. (www.tekscan.com). This sensor is described in detail in U.S. Pat. No. 6,272,936, the contents of which are incorporated herein by reference. During operation, the sensor 34 contacts skin disposed directly above an underlying artery in the patient's wrist, and measures a change in pressure caused by each heartbeat. To accurately characterize pressure, a data-processing circuit 32, embedded in the wrist-worn module, passes current through the pressure sensor 34. This results in a voltage that varies with the pressure-sensitive electrical resistance. The analog-to-digital converter 46 samples the variable voltage and in response generates a time-dependent pressure waveform that the data-processing circuit 32 receives, stores in an internal memory, and then analyzes. Specifically, the circuit 32 includes a microprocessor that runs computer-readable firmware to analyze both the optical and pressure waveforms using one of the algorithms described in detail below. Processing these waveforms yields blood pressure, pulse oximetry, heart rate, along with various statistics (e.g., average values, standard deviation) of this information.

Once determined, the data-processing circuit 32 sends the calculated values and waveforms to an LCD 42 seated on the wrist-mounted module. The LCD 42 displays this information, as well as text messages sent from the Internet-accessible website. Additionally the circuit 32 avails the calculated values and waveforms through a serial port 40 to a personal computer, which displays and analyzes the information using a client-side software application. A battery 37 powers all the electrical components within the watch component, and is typically a metal hydride battery (typically generating 5V) that can be recharged through a battery recharge interface 44.

In order to transmit information to the wireless hub, the wrist-mounted module includes a wireless, short-range wireless transmitter 38 (e.g., a Bluetooth™ transmitter) that receives information from the data-processing circuit 32 and transmits this information in the form of a packet through an antenna 39. A matched antenna 49 coupled to a wireless, short-range receiver 50 (e.g., a Bluetooth™ receiver) in the wireless hub receives the packet and passes it to a microprocessor 45. The microprocessor 45 formats the information in a packet suitable for transmission through the wireless network, and then sends the packets to a long-range wireless modem 41 (e.g., a modem operating on the Mobitex or DataTac networks). Using an antenna 43, the long-range wireless modem 41 transmits the packet through the wireless network to an Internet-accessible website.

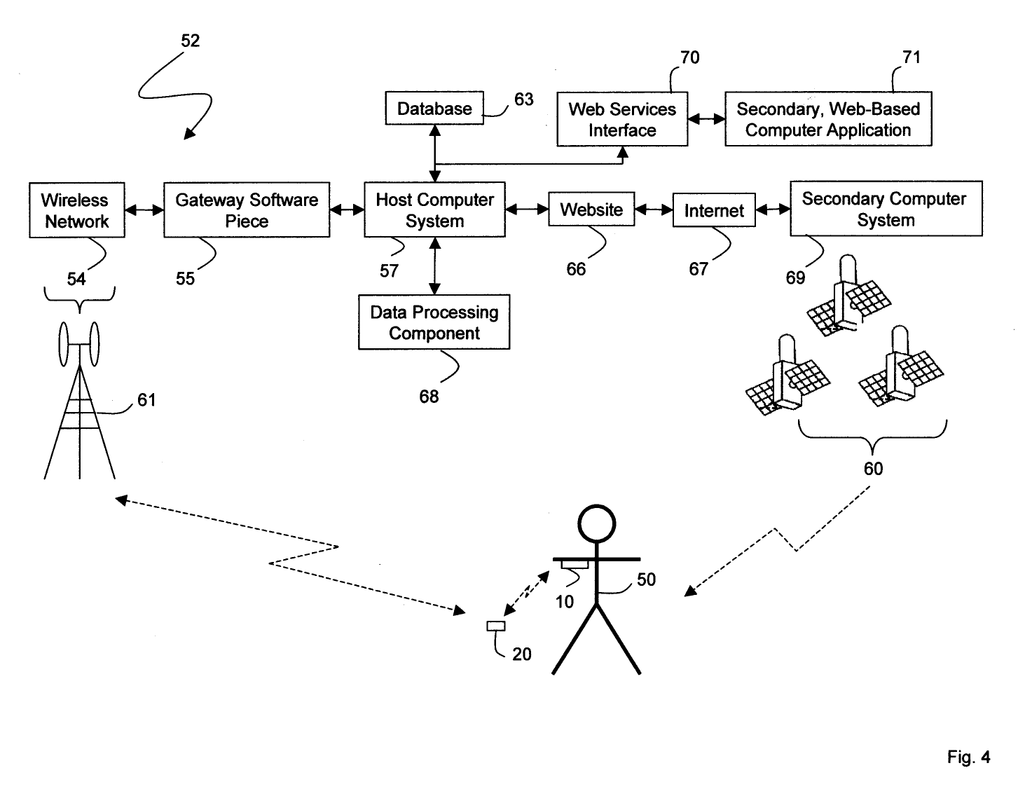

FIG. 4 shows an Internet-based system 52 that operates in concert with the watch component 10 and wireless hub 20 to send information from a patient 50 through a wireless network to the Internet. During operation, the wireless hub 20 transmits this information over a two-way wireless network 54 and ultimately to a web site 66. A secondary computer system 69 accesses the website 66 through the Internet 67. The system 52 functions in a bi-directional manner, i.e. the wireless hub 20 can both send and receive data. Most data flows from the hub 20; using the same network, however, this module also receives data (e.g., “requests” to measure data or text messages) and software upgrades.

Data are typically transmitted through the wireless network 54 as packets that feature a “header” and a “payload”. The header includes an address of the source wireless transmitter and a destination address on the network. The payload includes the above-described data. Data packets are transmitted over conventional wireless terrestrial network, such as a CDMA, GSM/GPSRS, Mobitex, or DataTac network. Or they may be transmitted over a satellite network, such as the Orbcomm network. The specific network is associated with the wireless transmitter used by the monitor to transmit the data packet.

A gateway software piece 55 connects to the wireless network 54 and receives the data packet from one or more devices. The gateway software piece 55 additionally connects to a host computer system 57 that includes a database 63 and a data-processing component 68 for, respectively, storing and analyzing the data. The host computer system 57, for example, may include multiple computers, software pieces, and other signal-processing and switching equipment, such as routers and digital signal processors. The gateway software piece 55 typically connects to the wireless network 54 using a TCP/IP-based connection, or with a dedicated, digital leased line (e.g., a frame-relay circuit or a digital line running an X.25 protocol). The host computer system 57 also hosts the web site 66 using conventional computer hardware (e.g. computer servers for both a database and the web site) and software (e.g., web server and database software).

During typical operation, the patient continuously wears the blood-pressure monitor for a short period of time, e.g. one to two weeks after visiting a medical professional during a typical “check up” or after signing up for a short-term monitoring program through the website. For longer-term monitoring, the patient may measure blood pressure once each day for several months. To view information sent from the blood-pressure monitor, the patient or medical professional accesses a patient user interface hosted on the web site 66 through the Internet 67 from a secondary computer system 69. The patient interface displays blood pressure and related data measured from a single patient. The system 52 may also include a call center, typically staffed with medical professionals such as doctors, nurses, or nurse practitioners, who access a care-provider interface hosted on the same website 66. The care-provider interface displays blood pressure data from multiple patients.

In an alternate embodiment, the host computer system 57 includes a web services interface 70 that sends information using an XML-based web services link to a secondary, web-based computer application 71. This application 71, for example, could be a data-management system operating at a hospital.

Referring to FIGS. 3 and 4, the wrist-worn component 10 may additionally include a GPS 47 that receives GPS signals through an antenna 48 from a constellation of GPS satellites 60 and processes these signals to determine a location (e.g., latitude, longitude, and altitude) of the monitor and, presumably, the patient. This location could be used to locate a patient during an emergency, e.g. to dispatch an ambulance.

The steps for processing the pressure and pulse-oximetry waveforms to determine blood pressure are described in detail in a co-pending patent application, filed on the same day as this application, entitled CUFFLESS SYSTEM FOR MEASURING BLOOD PRESSURE, the contents of which are incorporated herein by reference.

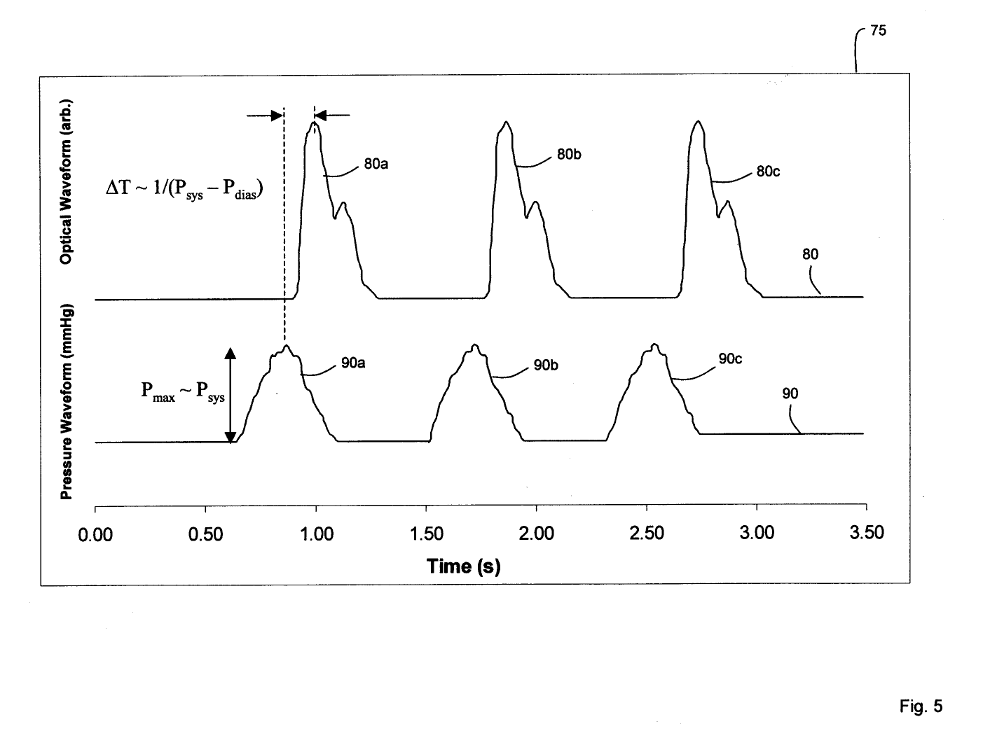

FIG. 5 shows a graph 75 that indicates how the microprocessor within the wrist-mounted component processes optical 80 and pressure 90 waveforms to determine blood pressure. During a measurement, the watch component 10 is worn with the wrist-worn module secured to the patient's wrist (like a watch), and the finger-worn module secured to the patient's finger (like a ring). Blood flowing following a heartbeat causes pressure in an underlying artery to rise from the diastolic pressure (P_{dias}) to the systolic pressure (P_{sys}). The thin-film pressure sensor within sys the wrist-mounted component detects a pressure waveform 90, with each heartbeat generating a “pressure pulse” 90a-c with a magnitude indicating a heartbeat-induced rise in pressure. This pressure rise, as shown in FIG. 5, is proportional to the systolic pressure. Blood flowing through the artery from the wrist to the finger is measured at a later time by the optical module within the finger-mounted module. The module generates the optical waveform 80 featuring a series of “optical pulses” 80a-c, like the pressure pulses 90a-c, each corresponding to an individual heartbeat.

The time difference between when the thin-film pressure sensor measures a pressure pulse and when the optical module measures a corresponding optical pulse is the time it takes blood to flow along a length ΔL of the artery. This time, shown in FIG. 5 as ΔT, yields the flow rate (ΔT=1/Q˜1/(P_{sys}−P_{dias})). The microprocessor calculates ΔT by measuring the peak intensity of both the optical and pressure pulses, and then calculating the time lag between these pulses.

A calibration process is typically required to convert Q into a pressure value using the equation:
ΔP=16νΔLQ/r² (1)

This simplified equation considers the artery to be elastic and the flow of blood to be pulsatile, i.e. not steady state, and takes into account Poiseuillei″s law, which describes a Newtonian liquid propagating in a tube. According to Poiseuillei″s law, the linear flow (Q) through a tube of length L and radius r relates to a pressure gradient (ΔP) and the viscosity (ν) of the flowing liquid (i.e. blood).

To calibrate the watch component, a patient attaches a stand-alone cuff to their arm prior to making an actual measurement. The cuff features a serial output that sends pressure values to the watch component as the cuff inflates. This cuff is only used during calibration. To ‘set up’ the system, the user inflates the cuff, which in turn applies pressure to the arm and underlying artery. Pressure gradually increases until it first meets the patient's diastolic pressure. At this point, the cuff compromises blood flow in the artery, and the pulses in the optical waveform begin to decrease. This determines P_{dias}. As the pressure increases to the systolic pressure, the signal measured by both the thin-film pressure sensor and the optical module decrease to 0. This is because temporarily stops flowing through the artery because of the applied pressure, and thus no signals are measured. This determines P_{sys}. The patient then removes the cuff, at which point the watch component begins measuring ΔT (and thus Q).

With these values, Eqn. 1 reduces to:
ΔP=P_{sys}−P dias =X₁Q (2)
where X₁ is a calibration factor that accounts for blood viscosity (ν), the radius of the underlying artery (r), and the length separating the pressure sensor and optical module (ΔL). Using X₁, the microprocessor analyzes a simple measurement of ΔT to determine ΔP=P_{sys}−P_{dias}. In addition, the calibration process can be used to correlate the maximum pulse magnitude in the pressure waveform to P_{sys}:
Pₘₐₓ=X₂P_{sys} (3)

The calibration factors X₁, X₂ are automatically calculated by the microprocessor during the set-up process and used for all on-going measurements.

Once the calibration is performed, the cuff is removed, and the watch component measures flow rate to determine systolic and diastolic pressure using the calibration factors as described above. Measurements can be performed continuously without any discomfort to the patient because no cuff is required.

The monitor determines beat-by-beat blood pressure by processing the systolic and diastolic blood pressures determined as described above with an optical waveform, similar to that shown in FIG. 5. This processing involves a simple linear transformation wherein the baseline of the optical waveform is mapped to the diastolic pressure, and the average height of a train of pulses is mapped to the systolic pressure. The linear transformation algorithm determines points in between these two extremes.

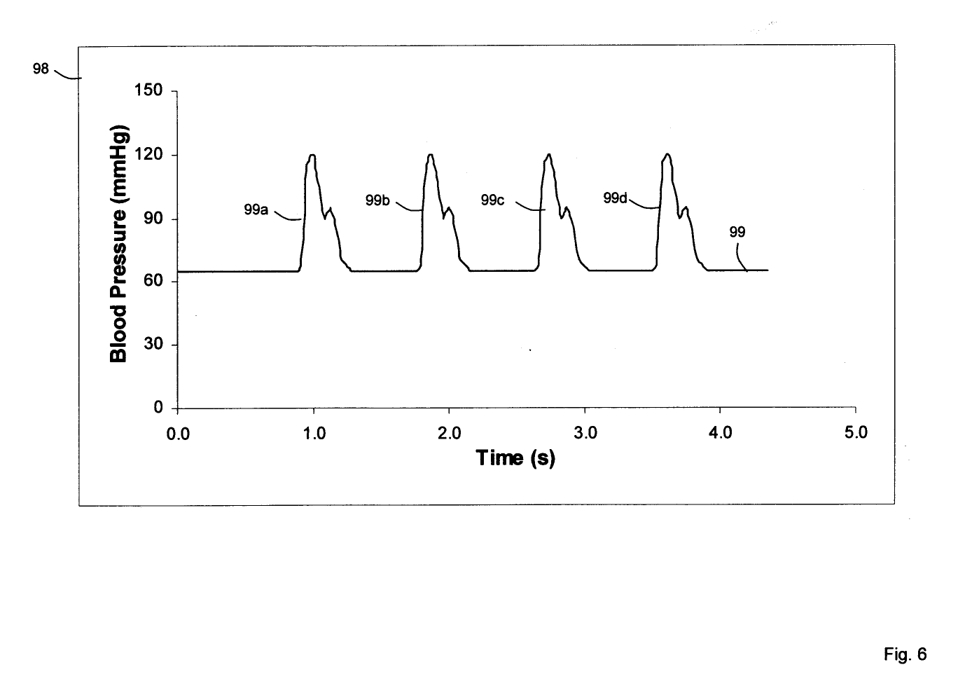

FIG. 6 shows a graph 98 that plots the beat-to-beat blood pressure resulting from the above-described measurements. The graph 98 features a waveform 99, indicating the patient's real-time, beat-to-beat blood pressure. The waveform 99 includes a baseline that represents the diastolic blood pressure (in this case about 66 mmHg). As the patient's heart beats, blood volume forces through the measured artery, increasing the blood pressure. A first pulse 99a in the waveform 99 indicates this increase. The maximum value of the pulse (in this case about 117 mmHg) represents the systolic blood pressure. As the blood volume passes through the artery, the pressure decreases and returns to the baseline, diastolic value. This cycle is repeated, as represented by additional pulses 99b-d, as the patient's heart continues to beat.

Other embodiments are within the scope of the invention. For example, the placement of the above-described optical, mechanical, and electrical modules can be modified to change the form factor of the device. Or the modules can be integrated into a single hand-held device or an arm-worn patch. Other configurations of the above-described optical, mechanical, and electrical sensors are also within the scope of the invention.

The watch component can also use algorithms other than those described above to process data measured by the module. These algorithms are typically based on the equations described above, but may vary in their form. In other embodiments, electrical components within the watch component (as shown in FIG. 3) are consolidated into a single silicon-based device.

The device can also be used in ways other than those described above. For example, in one embodiment, a patient using an Internet-accessible computer and web browser, such as those described in FIG. 4, directs the browser to an appropriate URL and signs up for a service for a short-term (e.g., 1 month) period of time. The company providing the service completes an accompanying financial transaction (e.g. processes a credit card), registers the patient, and ships the patient a blood-pressure monitor for the short period of time. The registration process involves recording the patient's name and contact information, a number associated with the monitor (e.g. a serial number), and setting up a personalized website. The patient then uses the monitor throughout the monitoring period, e.g. while working, sleeping, and exercising. During this time the monitor measures data from the patient and wirelessly transmits it through the channel described in FIG. 4 to a data center. There, the data are analyzed using software (e.g., reporting software supported by an Oracle™ database) running on computer servers to generate a statistical report. The computer servers then automatically send the report to the patient using email, regular mail, or a facsimile machine at different times during the monitoring period. When the monitoring period is expired, the patient ships the blood-pressure monitor back to the monitoring company.

In other embodiments, the watch component includes an electrical impedance (EI) sensor that features an electrode pair that characterizes impedance plethysmography as a way of determining changing tissue volumes in an underlying tissue body. The EI sensor measures electric impedance at the tissue surface by transmitting a small amount of alternating current (typically between 20-100 kHz) through the underlying tissue. The tissue includes components such as bone and skin that have a static (i.e. time invariant) impedance, and flowing blood, which has a dynamic (i.e. time varying) impedance. Blood has a well-defined resistivity of about 160 Ω-cm. Impedance, defined as electrical resistance to alternating current, will therefore vary as the volume of blood in the tissue changes with each heartbeat. Measurements made with the EI sensor, following processing with a firmware algorithm, yield an impedance waveform that features “pulses” indicating the time-dependent volumetric flow of blood. When the EI sensor replaces the thin-film pressure sensor, the separation between pulses in the impedance waveform and those in the optical waveform yield ΔP. Combined with the above-described calibration process, the magnitude of each pulse can be correlated to P_{sys}. The entire impedance waveform can therefore be used in place of the pressure waveform to determine P_{sys} and P_{dias}.

In addition to this sensor, the blood-pressure monitor can include a pair of optical modules that measure the time-dependent variation in arterial diameter caused by blood flow. These data, along with data generated by the EI sensor, can be processed with a mathematical algorithm to determine blood pressure.

The mathematical algorithm used for this calculation can take many forms. For example, the paper entitled “Cuffless, Continuous Monitoring of Beat-to-Beat Pressure Using Sensor Fusion” (Boo-Ho Yang, et al., submitted to the IEEE Transactions on Biomedical Engineering, 2000) describes an algorithm based on a two-dimensional Navier-Stokes differential equation that models pulsatile flow of a Newtonian liquid (e.g., blood) through an elastic, deformable cylindrical vessel (e.g., an artery). This differential equation can be solved in a number of different ways to determine the patient's blood pressure.

In other embodiments, the watch component includes a pair of optical modules, as described above, measure blood flow at two separate points on a patient. A microprocessor processes these data to determine a time difference (ΔT) for blood to flow from the first point to the second point. The microprocessor detects the separation between the peak values of two sequential pulses and uses an internal real-time clock to convert this separation into a time value. These parameters are then processed according to the algorithm and calibration process described below to determine blood flow rate that is then used to determine the systolic and diastolic pressures.

In still other embodiments, the antennae used to transmit the blood pressure data or receive the GPS signals are embedded in the monitor, rather than being exposed.

Web pages used to display the data can take many different forms, as can the manner in which the data are displayed. Web pages are typically written in a computer language such as “HTML” (hypertext mark-up language), and may also contain computer code written in languages such as java and javascript for performing certain functions (e.g., sorting of names). The web pages are also associated with database software (provided by companies such as Oracle and Microsoft) that is used to store and access data. Equivalent versions of these computer languages and software can also be used. In general, the graphical content and functionality of the web pages may vary substantially from what is shown in the above-described figures. In addition, web pages may also be formatted using standard wireless access protocols (WAP) so that they can be accessed using wireless devices such as cellular telephones, personal digital assistants (PDAs), and related devices.

Different web pages may be designed and accessed depending on the end-user. As described above, individual users have access to web pages that only their blood pressure data (i.e., the patient interface), while organizations that support a large number of patients (e.g. hospitals) have access to web pages that contain data from a group of patients (i.e., the care-provider interface). Other interfaces can also be used with the web site, such as interfaces used for: insurance companies, members of a particular company, clinical trials for pharmaceutical companies, and e-commerce purposes. Blood pressure data displayed on these web pages, for example, can be sorted and analyzed depending on the patient's medical history, age, sex, medical condition, and geographic location.

The web pages also support a wide range of algorithms that can be used to analyze data once they are extracted from the data packets. For example, an instant message or email can be sent out as an “alert” in response to blood pressure indicating a medical condition that requires immediate attention. Alternatively, the message could be sent out when a data parameter (e.g. systolic blood pressure) exceeds a predetermined value. In some cases, multiple parameters (e.g., blood pressure and pulse oximetry) can be analyzed simultaneously to generate an alert message. In general, an alert message can be sent out after analyzing one or more data parameters using any type of algorithm. These algorithms range from the relatively simple (e.g., comparing blood pressure to a recommended value) to the complex (e.g., predictive medical diagnoses using “data mining” techniques). In some cases data may be “fit” using algorithms such as a linear or non-linear least-squares fitting algorithm. In general, any algorithm that processes data collected with the above-described method is within the scope of the invention.

Still other embodiments are within the scope of the following claims.

1. A blood-pressure monitoring device, comprising:
a thin-film, pressure-monitoring module comprising a pressure-sensitive region;
an optical module comprising an optical source that generates both red and infrared radiation and an optical transmission detector; and
a microprocessor configured to receive and process information from the thin-film, pressure-monitoring module and the optical module to determine blood pressure.

2. The blood-pressure monitoring device of claim 1, wherein the pressure-sensitive region comprises a material characterized by pressure-dependent electrical properties.

3. The blood-pressure monitoring device of claim 1, wherein the pressure-monitoring module comprises a plastic film that encases the pressure-sensitive region.

4. The blood-pressure monitoring device of claim 1, wherein the optical source comprises a laser or a light-emitting diode.

5. The blood-pressure monitoring device of claim 1, wherein the optical detector is comprises a photodiode.

6. The blood-pressure monitoring device of claim 1, further comprising a finger-mounted component that comprises the optical module.

7. The blood-pressure monitoring device of claim 6, wherein the finger-mounted component is an annular ring.

8. The blood-pressure monitoring device of claim 1, further comprising a wrist-mounted component that comprises the thin-film pressure-monitoring module.

9. The blood-pressure monitoring device of claim 1, further comprising a short-range wireless transmitter.

10. The blood-pressure monitoring device of claim 9, wherein the short-range wireless transmitter is a radio-frequency transmitter operating a peer-to-peer, part-15, or 802.11 wireless protocol.

11. The blood-pressure monitoring device of claim 1, further comprising an external, secondary wireless component.

12. The blood-pressure monitoring device of claim 11, wherein the external, secondary wireless component comprises a short-range wireless receiver.

13. The blood-pressure monitoring device of claim 12, wherein the short-range wireless receiver is a radio-frequency receiver operating a peer-to-peer, part-15, or 802.11 wireless protocol.

14. The blood-pressure monitoring device of claim 11, wherein the external, secondary wireless component further comprises a long-range wireless transmitter.

15. The blood-pressure monitoring device of claim 14, wherein the long-range wireless transmitter is configured to transmit information over a terrestrial, satellite, or 802.11-based wireless network.

16. The blood-pressure monitoring device of claim 15, wherein the long-range wireless transmitter is configured to transmit data over a wireless network operating on at least one of the following protocols: CDMA, GPRS, and analogs and derivatives thereof.

17. The blood-pressure monitoring device of claim 1, wherein the pressure-monitoring module is configured to generate a pressure waveform.

18. The blood-pressure monitoring device of claim 17, wherein the optical module is configured to generate an optical waveform.

19. The blood-pressure monitoring device of claim 18, wherein the microprocessor comprises computer-readable code that processes both the optical and pressure waveforms to determine blood pressure.

20. A blood pressure monitoring device, comprising:
an optical sensor for measuring the transmission of light at two different wavelengths through a person's finger;
a thin-film pressure sensor for measuring pressure above an underlying artery in a person's wrist;
a microprocessor configured to receive and process information from the thin-film pressure sensor and the optical sensor for determining blood pressure; and,
a short-range wireless transmitter for transmitting blood pressure information to a wireless hub.
